(19) United States
(12) Patent Application Publication
(10) Pub. No.: US 2013/0195776 A1
(43) Pub. Date: Aug. 1, 2013

(54) COSMETIC COMPOSITION BASED ON ELLAGIC ACID OR A DERIVATIVE THEREOF AND A BACTERIAL EXTRACT

(71) Applicant: Tiphaine Derkx, Paris (FR)

(72) Inventor: Tiphaine Derkx, Paris (FR)

(73) Assignee: L'OREAL S.A., Paris (FR)

(21) Appl. No.: 13/581,638

(22) PCT Filed: Feb. 28, 2011
(86) PCT No.: PCT/EP2011/052934

§ 371 Date: Aug. 29, 2012

Related U.S. Application Data
(60) Provisional application No. 61/309,539, filed on Mar. 2, 2010.

(30) Foreign Application Priority Data
Mar. 1, 2010 (FR) 1051448

(51) Int. Cl. A61K 8/49; A61Q 17/04; A61Q 5/00; A61K 8/99

(52) U.S. Cl. CPC A61K 8/498; A61K 8/99; A61Q 17/04; A61Q 5/006
USPC 424/59; 424/780

(57) ABSTRACT

The invention relates to a cosmetic composition comprising, in a cosmetically acceptable medium: (i) from 0.01% to 10% by weight, relative to the total weight of the composition, of one or more compounds chosen from ellagic acid, ethers thereof and ellagic acid salts or ethers thereof, in a content ranging (ii) one or more extracts of one or more non-photosynthetic, non-fruiting filamentous bacteria, and (iii) one or more surfactants chosen from anionic and/or amphoteric or zwitterionic surfactants. The invention also relates to a cosmetic treatment process using such a composition, and to the use of this composition for eliminating and/or reducing the amount of dandruff on the hair and the scalp.

The present invention relates to a cosmetic composition comprising at least a first compound chosen from ellagic acid, ethers thereof and salts of these compounds, and at least a second particular compound, to an antidandruff treatment process using the said composition, and to the use of the said composition for treating dandruff conditions without bringing about in particular irritation and/or itching of the scalp. In particular, the invention also relates to the use of the combination of at least a first compound chosen from ellagic acid, ethers thereof and salts thereof, and of at least a second particular compound, as an antidandruff agent.

Dandruff problems affect up to 50% of the world's population. They affect both men and women and are perceived as having a very negative psychosocial impact. The appearance of dandruff is disagreeable both aesthetically and because of what it brings about (itching, redness, etc.), and as such many people confronted with this problem to variable degrees wish to eliminate it efficiently and definitively.

Dandruff corresponds to excessive and visible desquamation of the scalp resulting from excessively rapid multiplication of the epidermal cells and their abnormal maturation. This phenomenon may be caused especially by microtrauma of physical or chemical nature, such as excessively aggressive hair treatments, extreme climatic conditions, nervousness, the diet, fatigue and pollution, but it has been demonstrated that dandruff conditions usually result from a disorder of the microflora of the scalp and are more particularly due to the excessive colonization of a fungus belonging to the genus Malassezia (previously known as Pityrosporum) which is naturally present on the scalp.

To treat dandruff, it is known practice to use antidandruff agents, especially antifungal and/or antibacterial agents in a medium capable of distributing these agents and depositing them on the teguments.

Among antidandruff agents, zinc pyrithione, piroctone ylamine and selenium disulfide have been most particularly recommended on account of their powerful cytostatic activity. Although they show good antidandruff efficacy, their frequent use has the drawback of accentuating the discomfort sensations (itching, stinging, heating, redness or dryness sensations). Moreover, these antidandruff agents are not entirely satisfactory in terms of their environmental impact.

Thus, there is a need, on the part of users of antidandruff treatments, for compositions that show better skin tolerance, while at the same time conserving good antidandruff efficacy and being more environmentally friendly. In addition, antidandruff compositions that are stable and that have good working properties, especially during the application or removal of the compositions, are sought.

The antidandruff activity of non-fruiting non-photosynthetic bacterial extracts is also known (FR 2 879 452).

However, although non-fruiting non-photosynthetic bacterial extracts have good antidandruff activity, they nevertheless have the drawback of not acting on the related nuisances such as stinging, heating sensations and redness.

Compositions that do not have the drawbacks mentioned above and that have reinforced antidandruff activity are sought.

The Applicant has now found, surprisingly, that the use of at least a first compound chosen from ellagic acid, ethers thereof, and ellagic acid salts or ethers thereof, and of at least one non-fruiting non-photosynthetic filamentous bacterial extract makes it possible to satisfy such a need, and to overcome the drawbacks of the prior art.

The composition according to the invention makes it possible to efficiently eliminate and/or reduce dandruff on the hair and the scalp, in particular dandruff caused by yeasts of the genus Malassezia, while at the same time notably reducing the irritation and itching of the scalp.

It also does not alter the cosmetic properties of the hair treated in conjunction, especially in terms of sheen, suppleness and smoothness of the hair.

Moreover, surprisingly, the Applicant has discovered that the composition of the invention has a particularly pronounced anti-canities effect, i.e. it can even more efficiently combat greying of the hair when compared with the constituents taken individually.

One subject of the present invention is thus a cosmetic composition comprising:

(i) one or more compounds chosen from ellagic acid, ethers thereof and ellagic acid salts or ethers thereof, and

(ii) one or more extracts of one or more non-photosynthetic, non-fruiting filamentous bacteria.

Another subject of the invention relates to a cosmetic antidandruff treatment process, for eliminating and/or reducing dandruff, in particular dandruff caused by yeasts of the genus Malassezia, using the said composition.

A subject of the invention is also the use of the said composition for treating dandruff conditions, that is to say for eliminating and/or reducing the amount of dandruff on the hair and the scalp, and for eliminating and/or reducing the irritation and/or itching of the scalp.

Other features, aspects, objects and advantages of the present invention will emerge even more clearly from a reading of the description and examples which follow.

The composition according to the invention comprises one or more compounds (i) chosen from ellagic acid, ethers thereof and ellagic acid salts or ethers thereof.

Ellagic acid, also known as: 2,3,7,8-tetrahydroxy(1)-benzopyrano(5,4,3-cde)(1)benzopyran-5,10-dione, is a well-known molecule belonging to the polyphenol group, and is present in the plant kingdom. Reference may be made to the Merck Index 20th edition (1996), No. 3588.

Ellagic acid has the following chemical formula:

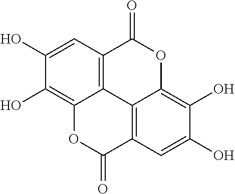 [SMILES: O=c1oc2c(O)c(O)cc3c(=O)oc4c(O)c(O)cc1c4c23]

which comprises four fused rings.

Ellagic acid is commercially available, especially from the company Sigma, France.

Document FR-A-1 478 523 discloses a process for purifying ellagic acid and also the purified ellagic acids obtained via such a process.

The ellagic acid ether(s) are preferably chosen from the mono-, di-, tri- or polyethers obtained by etherification of one or more hydroxyl groups (one of the four OH groups of ellagic acid) with one or more groups chosen from C₂-C₂₀ alkyl groups, polyoxyalkylene groups or groups derived from one or more mono- or polysaccharides.

Such ethers are described in U.S. Pat. No. 5,073,545. Preferably, the ellagic acid ethers are chosen from 3,4-di-O-methylellagic acid, 3,3′,4-tri-O-methylellagic acid and 3,3′-di-O-methylellagic acid.

The ellagic acid salt(s) that may be used according to the invention are preferably chosen from the salts of alkali metals or alkaline-earth metals, such as sodium, potassium, calcium and magnesium, ammonium salts and amine salts such as triethanolamine, monoethanolamine, arginine and lysine salts. Preferably, the ellagic acid salt(s) or ethers thereof that may be used according to the invention are chosen from the salts of alkali metals or alkaline-earth metals, especially the sodium, potassium, calcium or magnesium salts.

Among all the compounds (i) mentioned, it is preferred to use ellagic acid and salts thereof.

The composition according to the invention preferably comprises from 0.01% to 10% by weight, in particular from 0.1% to 5% by weight and better still from 0.2% to 2% by weight of compound(s) (i), relative to the total weight of the composition.

The bacterial extracts that may be used according to the invention will be chosen from non-photosynthetic, non-fruiting filamentous bacteria as defined according to the classification in Bergey's Manual of Systemic Bacteriology, volume 3, section 23, 9^{th} edition 1989.

Among the bacteria that may be used, mention will be made more particularly of bacteria belonging to the order Beggiatoales, and especially bacteria belonging to the genus Beggiotoa, for instance various strains of Beggiotoa alba. According to the definition, B. alba corresponds to the former names Beggiotoa arachnoidea, B. gigantea, B. leptomiformis, B. minima and B. mirabilis of Bergey's manual, 8th edition. Mention may moreover be made of bacteria belonging to the genus Vitreoscilla, which is known to be close to and often difficult to distinguish from the genus Beggiatoa. The bacteria that have just been defined, and several of which have been described, generally have an aquatic habitat, and may be found especially in spring water sources.

Among the bacteria that may be used, mention may be made, for example, of Vitreoscilla beggiatoides (ATCC 43181) and Beggiatoa alba (ATCC33555).

Preferentially, according to the invention, the use of the extract of Vitreoscilla filiformis, in particular the strain ATCC 15551, metabolites thereof and fractions thereof, are claimed.

Moreover, it is known that culturing non-photosynthetic, non-fruiting filamentous bacteria is relatively difficult, as is the production of pure cultures. Use will preferentially be made of the culture described in patent application WO 94/02158.

The term “non-photosynthetic, non-fruiting filamentous bacteria” means not only the culture supernatant but also the biomass obtained after culturing the said bacteria, the envelopes or envelope fractions, or the extracts of the biomass obtained by treating this biomass.

To prepare the extract according to the invention, the said bacteria can be cultured and then separated from the biomass obtained, for example by filtration, centrifugation, coagulation and/or lyophilization.

The extracts that may be used may especially be prepared according to the process described in patent application WO-A-93/00741. Thus, after culturing, the bacteria are concentrated by centrifugation. The biomass obtained is autoclaved. This biomass may be lyophilized to constitute what is known as the lyophilized extract. Any lyophilization method known to those skilled in the art may be used to prepare this extract.

The supernatant fraction of this biomass may also be filtered in a sterile container to remove the particles in suspension.

The terms “envelopes” and “envelope fractions” refer herein to the bacterial wall and possibly the subjacent membranes.

The bacterial extract(s) (ii) may be used in a concentration of from 0.001% to 10% by weight, from 0.01% to 5% by weight, preferably from 0.05% to 5% by weight and more particularly from 0.1% to 3% by weight expressed as solids relative to the total weight of the composition.

The composition according to the invention is, generally, preferably the weight ratio of the amount of compound(s) such that the weight ratio of the amount of compound(s) (i) to the amount of extract(s) (ii) is greater than or equal to 0.5.

Even more preferentially, the weight ratio of the amount of compound(s) (i) to the amount of extract(s) (ii) is greater than or equal to 0.7 and better still greater than or equal to 1.

Preferably, (I) to the amount of extract(s) (ii) is less than or equal to 50, better still less than or equal to 20, even better still less than or equal to 10 and more particularly less than 5.

The composition according to the invention may also comprise one or more thickeners.

For the purposes of the present invention, the term “thickener” means an agent which, when introduced at 1% by weight in an aqueous solution or an aqueous-alcoholic solution containing 30% ethanol, and at pH 7, makes it possible to achieve a viscosity of at least 100 cps and preferably of at least 500 cps, at 25° C. and at a shear rate of 1 s⁻¹. This viscosity may be measured using a cone/plate viscometer (Haake R600 rheometer or the like).

The thickener(s) may be chosen from sodium chloride, fatty acid amides obtained from C10-C30 carboxylic acids (coconut acid monoisopropanol-, diethanol- or monoethanol-amide, oxyethylenated carboxylic acid monoethanolamide alkyl ether), cellulose-based thickeners (hydroxyethycellulose, hydroxypropylcellulose, carboxymethylcellulose), guar gum and derivatives thereof (hydroxypropyl guar), gums of microbial origin (xanthan gum, scleroglucan gum), crosslinked homopolymers and copolymers based on acrylic acid or methacrylic acid or acrylamidopropanesulfonic acid and the associative polymers as described below.

The associative polymer(s) that may be used according to the invention are water-soluble polymers that are capable, in aqueous medium, of reversibly combining with each other or with other molecules.

Their chemical structure comprises hydrophilic zones and hydrophobic zones characterized by at least one fatty chain preferably comprising from 10 to 30 carbon atoms.

The associative polymer(s) that may be used according to the invention may be of anionic, cationic, amphoteric or nonionic type, such as the polymers sold under the names Pemulen TR1 or TR2 by the company Goodrich (INCI: Acrylates/C10-30 Alkyl Acrylate Crosspolymer), Salcare SC90 by the company Ciba, Aculyn 22, 28, 33, 44 or 46 by the company Röhm & Haas and Elfacos T210 and T212 by the company Akzo.

Among all the thickeners mentioned, use is preferably made of homopolymers and copolymers based on acrylic acid or methacrylic acid, which are preferably crosslinked (add the preference in B094751 FR) the fatty acid amides obtained from a C10-C30 carboxylic acid.

Preferably, the cosmetic composition comprises from 0.1% to 20% by weight and better still from 0.2% to 10% by weight of thickener(s) relative to the total weight of the composition.

The composition according to the invention may also comprise one or more surfactants chosen from anionic, nonionic and/or amphoteric or zwitterionic surfactants.

These surfactants are mentioned in particular in the CTFA (2004 edition) under the name surfactant-cleansing agent.

The term “anionic surfactant” means a surfactant comprising as ionic or ionizable groups only anionic groups. These anionic groups are preferably chosen from the groups CO₂H, CO₂⁻, SO₃H, SO₃⁻, OSO₃H, OSO₃⁻, O₂PO₂H, O₂PO₂H⁻, O₂PO₂²⁻.

As examples of anionic surfactants (ii) that may be used in the composition according to the invention, mention may be made of alkyl sulfates, alkyl ether sulfates, alkylamido ether sulfates, alkylarylpolyether sulfates, monoglyceride sulfates, alkylsulfonates, alkylamidesulfonates, alkylarylsulfonates, alpha-olefin sulfonates, paraffin sulfonates, alkylsulfosuccinates, alkylether sulfosuccinates, alkylamide sulfosuccinates, alkylsulfoacetates, acylsarcosinates, acylglutamates, alkylsulfosuccinamates, acylisethionates and N-acyltaurates, salts of alkyl monoesters of polyglycoside-polycarboxylic acids, acyllactylates, D-galactoside-uronic acid salts, alkyl ether carboxylic acid salts, alkyl aryl ether carboxylic acid salts, alkylamido ether carboxylic acid salts; and the corresponding non-salified form is of all these compounds; the alkyl and acyl groups of all these compounds comprising from 6 to 24 carbon atoms and the aryl group denoting a phenyl group.

These compounds may be oxyethylenated and then preferably comprise from 1 to 50 and more particularly from 1 to 10 ethylene oxide units.

The salts of C6-24 alkyl monoesters of polyglycoside-polycarboxylic acids may be chosen from C6-24 alkyl polyglycoside citrates, C6-24 alkyl polyglycosides tartrates and C6-24 alkyl polyglycoside sulfosuccinates.

When the anionic surfactant(s) (ii) are in salt form, they may be chosen from alkali metal salts such as the sodium or potassium salt and preferably the sodium salt, the ammonium salts, the amine salts and in particular amino alcohol salts or the alkaline-earth metal salts such as the magnesium salt.

Examples of amino alcohol salts that may especially be mentioned include the salts of mono-, di- and triethanolamine, the salts of mono-, di- or triisopropanolamine, and the salts of 2-amino-2-methyl-1-propanol, of 2-amino-2-methyl-1,3-propanediol and of tris(hydroxymethyl)aminomethane.

The alkali metal or alkaline-earth metal salts and in particular the sodium or magnesium salts are preferably used.

Among all the anionic surfactants (ii) mentioned, use is preferably made of alkyl(C6-24) sulfates, alkyl(C6-24) ether sulfates comprising from 1 to 50 ethylene oxide units, especially in the form of alkali metal, ammonium, and alkaline-earth metal salts, or a mixture of these compounds.

In particular, use is preferably made of alkyl(C12-20) sulfates, alkyl(C12-20) ether sulfates comprising from 2 to 10 ethylene oxide units, especially in the form of alkali metal, ammonium, amino alcohol and alkaline-earth metal salts, or a mixture of these compounds. Better still, use is made of sodium lauryl ether sulfate containing 2.2 mol of ethylene oxide.

When they are present, the amount of anionic surfactant(s) preferably ranges from 0.1% to 50% by weight and better still from 4% to 30% by weight relative to the total weight of the composition.

Examples of nonionic surfactants that may be used in the cosmetic composition according to the invention are described, for example, in the “Handbook of Surfactants” by M. R. PORTER, published by Blackie & Son (Glasgow and London), 1991, pp. 116-178. They are especially chosen from polyethoxylated, or polyglycerolated alcohols, alpha-diols, (C₁₋₂₀)alkylphenols and fatty acids, containing a fatty chain comprising, for example, from 8 to 18 carbon atoms, ethylene oxide or propylene oxide groups possibly ranging especially from 2 to 50, and the number of glycerol groups possibly ranging especially from 2 to 30.

Mention may also be made of copolymers of ethylene oxide and propylene oxide, oxyethylenated fatty acid esters of sorbitan, fatty acid esters of sucrose, optionally oxyalkylenated alkylpolyglycosides, alkylglucoside esters, N-alkyl glucamine and N-acylmethylglucamine derivatives, aldobionamides and amine oxides.

When they are present, the amount of the nonionic surfactant(s) preferably ranges from 0.01% to 20% by weight and better still from 0.2% to 10% by weight relative to the total weight of the composition.

The amphoteric or zwitterionic surfactant(s) that may be used in the present invention may especially be derived from secondary or tertiary aliphatic amines, optionally quaternized, in which the aliphatic group is a linear or branched chain comprising from 8 to 22 carbon atoms, the said amine derivatives containing at least one anionic group, for instance a carboxylate, sulfonate, sulfate, phosphate or phosphonate group. Mention may be made in particular of alkyl(C₈₋₂₀)betaines, sulfobetaines, (C₈₋₂₀ alkyl)amido(C₂₋₈ alkyl)betaines and (C₈₋₂₀ alkyl)amido(C₂₋₈ alkyl)sulfobetaines.

Among the secondary or tertiary aliphatic amine derivatives, optionally quaternized, which may be used, as defined above, mention may also be made of the compounds having the respective structures (A1) and (A2)

Rₐ—CONHCH₂CH₂—N⁺R_{b})(R_{c})(CH₂COO⁻) (A1)

in which:

Rₐ represents a C10-C30 alkyl or alkenyl group derived from an acid Rₐ—COOH preferably present in hydrolysed coconut oil, or a heptyl, nonyl or undecyl group,

R_{b} represents a beta-hydroxyethyl group, and

R_{c} represents a carboxymethyl group;

and

Rₐ′—CONHCH₂CH₂—N(B)(B′) (A2)

in which:

B represents —CH₂CH₂OX′,

B′ represents —(CH₂)_{Z}—Y′, with z=1 or 2,

X′ represents the group —CH₂—COOH, CH₂—COOZ′, —CH₂CH₂—COOH, —CH₂CH₂—COOZ′, or a hydrogen atom,

Y′ represents —COOH, —COOZ′, the group —CH₂—CHOH—SO₃H or —CH₂—CHOH—SO₃Z′,

Z′ represents an ion derived from an alkali metal or alkaline-earth metal, such as sodium, an ammonium ion (NH4+) or an ion derived from an organic amine, in particular from amino alcohols.

Examples of amino alcohols that may especially be mentioned include mono-, di- and triethanolamine, mono-, di- and triisopropanolamine, 2-amino-2-methyl-1-propanol, 2-amino-2-methyl-1,3-propanediol and tris(hydroxymethyl)aminomethane.

Rₐ′ represents a C₁₀-C₃₀ alkyl or alkenyl group of an acid Rₐ′—COOH preferably present in coconut oil or in hydrolysed linseed oil, an alkyl group, especially of C₁₇ and its iso form, or an unsaturated C₁₇ group.

These compounds are classified in the CTFA dictionary, 5th edition, 1993, under the names disodium cocoamphodiacetate, disodium lauroamphodiacetate, disodium caprylamphodiacetate, disodium capryloamphodiacetate, disodium cocoamphodipropionate, disodium lauroamphodipropionate, disodium caprylamphodipropionate, disodium capryloamphodipropionate, lauroamphodipropionic acid and cocoamphodipropionic acid.

By way of example, mention may be made of the cocoamphodiacetate sold by the company Rhodia under the trade name Miranol® C2M Concentrate.

Among the amphoteric or zwitterionic surfactants mentioned above that are preferably used are (C₈₋₂₀ alkyl)betaines and (C₈₋₂₀ alkyl)amido(C₂₋₈ alkyl)betaines such as cocamidopropylbetaine, and mixtures thereof. More preferentially, the amphoteric or zwitterionic surfactants(s) are chosen from cocamidopropylbetaine and cocoylbetaine.

When they are present, the amount of the amphoteric or zwitterionic surfactants(s) is preferably within the range from 0.01% to 20% by weight and better still from 0.5% to 10% by weight relative to the total weight of the composition.

The composition according to the invention is generally used in topical application.

In particular, the composition according to the invention may be in any galenical form normally used for topical application.

The cosmetic composition used according to the invention may be a rinse-out or leave-in composition. In particular, it may be a shampoo, a cream, a mousse (aerosol or non-aerosol), a paste, a gel, an emulsion or a lotion, etc. Preferably, the cosmetic composition is a shampoo or a gel.

In a first preferred embodiment, the composition according to the invention is in the form of a gel. In this case, it comprises at least one thickener that is preferably present in an amount sufficient to obtain a gel.

The term “gel” or “gelled composition” means a composition with a viscosity ranging from 100 cps to 500 000 cps and better still from 200 cps to 100 000 cps at room temperature (25° C.) and at atmospheric pressure (1 bar) and at a shear rate of 1 s⁻¹. This viscosity may be measured using a cone/plate viscometer (Haake R600 rheometer or the like).

This particular form allows the composition to remain localized at the point of application. Thus, the composition does not run, which reduces the risks of contact of the composition with the eyes.

The thickener(s) may be chosen from those mentioned hereinabove.

In a second preferred embodiment, the composition according to the invention is in the form of a shampoo. In this case, the composition comprises one or more surfactants that are preferably present in an amount sufficient to wash the hair.

The surfactants(s) may be chosen from those defined previously.

Preferably, the total amount of anionic, nonionic, amphoteric and zwitterionic surfactants is then at least 3% by weight relative to the total weight of the composition. Even more preferentially, the composition according to the invention has a total content of anionic, nonionic, amphoteric and zwitterionic surfactants ranging from 4% to 50% by weight and better still from 4% to 20% by weight relative to the total weight of the composition.

The composition according to the invention generally comprises a cosmetically acceptable medium. The medium is formed from water and/or optionally one or more cosmetically acceptable organic solvents. Preferably, the medium is formed from water and optionally one or more cosmetically acceptable organic solvents.

The organic solvent(s) may be chosen from C₁-C₄ lower alcohols, such as ethanol, isopropanol, tert-butanol or n-butanol; polyols such as glycerol, propylene glycol and polyethylene glycols; polyol ethers, for instance dipropylene glycol monomethyl ether; and mixtures thereof.

Preferably, the cosmetic composition used according to the invention contains an amount of organic solvents ranging from 0.05% to 60%, preferably from 0.5% to 50% and better still from 1% to 40% by weight relative to the total weight of the composition.

The composition according to the invention may also comprise one or more conditioning agents.

According to the present invention, the term “conditioning agent” denotes any compound that can improve the cosmetic properties of the hair, in particular the softness, disentangling, feel and static electricity.

Preferably, the conditioning agent is chosen from the group comprising cationic polymers, cationic surfactants, silicones, in particular organosiloxanes, linear or branched C₈-C₃₀ hydrocarbons, linear or branched C₈-C₃₀ fatty alcohols, esters of a C₈-C₃₀ fatty acid and of a C₁-C₃₀ fatty alcohol, including esters of a C₈-C₃₀ fatty acid and of a C₈-C₃₀ fatty alcohol, esters of a C₁-C₇ acid or diacid and of a C₈-C₃₀ fatty alcohol, ceramides or ceramide analogues, and mixtures of these compounds.

The term “cationic polymer” means a polymer that is positively charged when it is contained in the composition according to the invention. This polymer may bear one or more positive permanent charges or may contain one or more cationizable functions in the composition according to the invention.

The cationic polymer(s) that may be used as conditioning agents according to the present invention are preferably chosen from polymers comprising primary, secondary, tertiary and/or quaternary amine groups forming part of the polymer chain or directly attached thereto, and having a molecular weight of between 500 and about 5 000 000 and preferably between 1000 and 3 000 000.

When the conditioning agent is a cationic polymer, it is preferably chosen from those that contain units comprising primary, secondary, tertiary and/or quaternary amine groups that may either form part of the main polymer chain, or may be borne by a side substituent directly attached thereto.

Among the cationic polymers that may be mentioned more particularly are polymers of the polyamine, polyamino amide and polyquaternary ammonium type. These are known products. They are described, for example, in French patents 2 505 348 and 2 542 997.

Among these polymers, mention may be made of:

(1) homopolymers or copolymers which are derived from acrylic or methacrylic esters or amides and comprise at least one of the units of the following formulae:

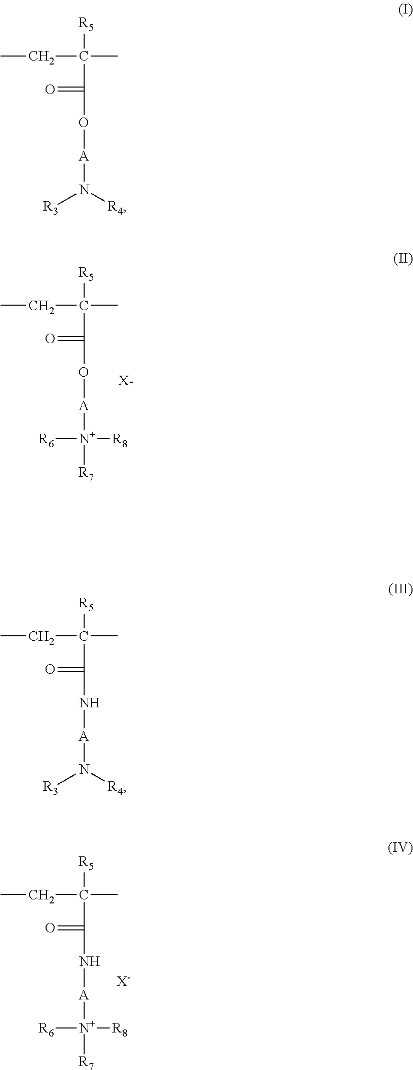

in which:

R3 and R4, which may be identical or different, represent hydrogen or an alkyl group containing from 1 to 6 carbon atoms and preferably methyl or ethyl;

R5, which may be identical or different, denote a hydrogen atom or a CH3 radical;

A, which may be identical or different, represents a linear or branched alkyl group of 1 to 6 carbon atoms, preferably 2 or 3 carbon atoms, or a hydroxyalkyl group of 1 to 4 carbon atoms;

R6, R7 and R8, which may be identical or different, represent an alkyl group containing from 1 to 18 carbon atoms or a benzyl radical and preferably an alkyl group containing from 1 to 6 carbon atoms;

X denotes an anion derived from a mineral or organic acid, such as a methosulfate anion or a halide such as chloride or bromide.

The polymers of family (1) can also contain one or more units derived from comonomers which may be chosen from the family of acrylamides, methacrylamides, diacetone acrylamides, acrylamides and methacrylamides substituted on the nitrogen with lower (C1-C4) alkyls, acrylic or methacrylic acids or esters thereof, vinyllactams such as vinylpyrrolidone or vinylcaprolactam, and vinyl esters.

Thus, among these copolymers of the family (1), mention may be made of:
copolymers of acrylamide and of dimethylaminoethyl methacrylate quaternized with dimethyl sulfate or with a dimethyl halide, such as the product sold under the name Hercofloc by the company Hercules,
the copolymers of acrylamide and of methacryloyloxy-ethyltrimethylammonium chloride described, for example, in patent application EP-A-080 976 and sold under the name Bina Quat P 100 by the company Ciba Geigy,
the copolymer of acrylamide and of methacryloyloxy-ethyltrimethylammonium methosulfate sold under the name Reten by the company Hercules,
quaternized or non-quaternized vinyl-pyrrolidone/dialkylaminoalkyl acrylate or methacrylate copolymers, such as the products sold under the name Gafquat by the company ISP, such as, for example, Gafquat 734 or Gafquat 755, or alternatively the products known as Copolymer 845, 958 and 937. These polymers are described in detail in French patents 2 077 143 and 2 393 573,
dimethylaminoethyl methacrylate/vinylcaprolactam-vinyl-pyrrolidone terpolymers, such as the product sold under the name Gaffix VC 713 by the company ISP,
vinylpyrrolidone/methacrylamidopropyldimethylamine copolymers sold in particular under the name Styleze CC 10 by ISP,
quaternized vinylpyrrolidone/dimethylaminopropyl-methacrylamide copolymers such as the product sold under the name Gafquat HS 100 by the company ISP, and
crosslinked polymers of methacryloyloxyalkyl(C1-C4)trialkyl(C1-C4)ammonium salts, such as the polymers obtained by homopolymerization of dimethylaminoethyl methacrylate quaternized with methyl chloride, or by copolymerization of acrylamide with dimethylaminoethyl methacrylate quaternized with methyl chloride, the homopolymerization or copolymerization being followed by crosslinking with an olefinically unsaturated compound, more particularly methylenebisacrylamide. A crosslinked acrylamide/methacryloyloxyethyltrimethylammonium chloride copolymer (20/80 by weight) in the form of a dispersion containing 50% by weight of the said copolymer in mineral oil can be used more particularly. This dispersion is sold under the name Salcare® SC 92 by the company Ciba. A crosslinked methacryloyloxyethyltrimethylammonium chloride homopolymer, for example as a dispersion in mineral oil or in a liquid ester, can also be used. These dispersions are sold under the names Salcare® SC 95 and Salcare® SC 96 by the company Ciba.

(2) Polymers formed from piperazinyl units and divalent alkylene or hydroxyalkylene radicals containing straight or branched chains, optionally interrupted with oxygen, sulfur or nitrogen atoms or with aromatic or heterocyclic rings, and also the oxidation and/or quaternization products of these polymers. Such polymers are especially described in French patents 2 162 025 and 2 280 361.

(3) Water-soluble polyamino amides prepared in particular by polycondensation of an acidic compound with a polyamine; these polyamino amides can be crosslinked with an epihalohydrin, a diepoxide, a dianhydride, an unsaturated dianhydride, a bisunsaturated derivative, a bis-halohydrin, a bis-azetidinium, a bis-haloacyldiamine, a bis-alkyl halide or alternatively with an oligomer resulting from the reaction of a difunctional compound which is reactive with a bis-halohydrin, a bis-azetidinium, a bis-haloacyldiamine, a bis-alkyl halide, an epihalohydrin, a diepoxide or a bis-unsaturated derivative; the crosslinking agent being used in proportions ranging from 0.025 to 0.35 mol per amine group of the polyamino amide; these polyamino amides can be alkylated or, if they contain one or more tertiary amine functions, they can be quaternized. Such polymers are especially described in French patents 2 252 840 and 2 368 508.

(4) Polyamino amide derivatives resulting from the condensation of polyalkylene polyamines with polycarboxylic acids followed by alkylation with difunctional agents. Mention may be made, for example, of adipic acid/dialkylaminohydroxyalkyl-dialkylene-triamine polymers in which the alkyl radical contains from 1 to 4 carbon atoms and preferably denotes methyl, ethyl or propyl. Such polymers are especially described in French patent 1 583 363.

Among these derivatives, mention may be made more particularly of the adipic acid/dimethylaminohydroxypropyl/diethylenetriamine polymers sold under the name Cartaretine F, F4 or F8 by the company Sandoz.

(5) The polymers obtained by reaction of a polyalkylene polyamine containing two primary amine groups and at least one secondary amine group with a dicarboxylic acid chosen from diglycolic acid and saturated aliphatic dicarboxylic acids having from 3 to 8 carbon atoms. The mole ratio between the polyalkylene polyamine and the dicarboxylic acid is between 0.8:1 and 1.4:1; the polyamino amide resulting therefrom is reacted with epichlorohydrin in a mole ratio of epichlorohydrin relative to the secondary amine group of the polyamino amide of between 0.5:1 and 1.8:1. Such polymers are described in particular in U.S. Pat. Nos. 3,227,615 and 2,961,347.

Polymers of this type are sold in particular under the name Hercosett 57 by the company Hercules Inc. or alternatively under the name PD 170 or Delsette 101 by the company Hercules in the case of the adipic acid/epoxypropyl/diethylenetriamine copolymer.

(6) Cyclopolymers of alkyldiallylamine or of dialkyldiallyl-ammonium, such as the homopolymers or copolymers containing, as main constituent of the chain, units corresponding to formula (V) or (VI):

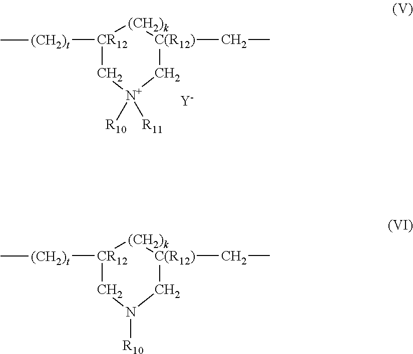

in which k and t are equal to 0 or 1, the sum k+t being equal to 1; R12 denotes a hydrogen atom or a methyl group; R10 and R11, independently of one another, denote an alkyl group having from 1 to 6 carbon atoms, a hydroxyalkyl group in which the alkyl group has preferably 1 to 5 carbon atoms, a lower (C1-C4) amidoalkyl group, or R10 and R11 may denote, jointly with the nitrogen atom to which they are attached, heterocyclic groups, such as piperidinyl or morpholinyl; Y⁻ is an anion such as bromide, chloride, acetate, borate, citrate, tartrate, bisulfate, bisulfite, sulfate or phosphate. These polymers are especially described in French patent 2 080 759 and in its Certificate of Addition 2 190 406.

R10 and R11, independently of each other, preferably denote an alkyl group having from 1 to 4 carbon atoms and more particularly 1 carbon atom.

Among the polymers defined above, mention may be made more particularly of the dimethyldiallylammonium chloride homopolymer sold under the name Merquat 100 by the company Nalco (and its homologues of low weight-average molecular mass) and the copolymers of diallyldimethylammonium chloride and of acrylamide, sold under the name Merquat 550.

(7) The quaternary diammonium polymer containing repeating units corresponding to the formula:

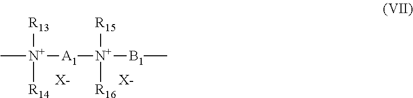

in which formula (VII):
R13, R14, R15 and R16, which may be identical or different, represent aliphatic, alicyclic or arylaliphatic groups containing from 1 to 20 carbon atoms, or lower hydroxyalkylaliphatic groups, or else R13, R14, R15 and R16, together or separately, constitute, with the nitrogen atoms to which they are attached, heterocycles optionally containing a second heteroatom other than the nitrogen, or else R13, R14, R15 and R16 represent a linear or branched C1-C6 alkyl group which is substituted by a nitrile, ester, acyl, amide or —CO—O—R17-D or —CO—NH—R17-D group in which R17 is an alkylene group and D is a quaternary ammonium group;

A1 and B1 represent polymethylene groups containing from 2 to 20 carbon atoms, which may be linear or branched, saturated or unsaturated, and which may contain, linked to or intercalated in the main chain, one or more aromatic rings or one or more oxygen or sulfur atoms or sulfoxide, sulfone, disulfide, amino, alkylamino, hydroxyl, quaternary ammonium, ureido, amide or ester groups, and
X— denotes an anion derived from a mineral or organic acid;
A1, R13 and R15 may form, with the two nitrogen atoms to which they are attached, a piperazine ring; moreover, if A1 denotes a saturated or unsaturated, linear or branched alkylene or hydroxyalkylene group, B1 may also denote a group (CH2)n-CO-D-OC—(CH2)p-,

n and p are integers ranging from 2 to 20 approximately
in which D denotes:
a) a glycol residue of formula: —O—Z—O—, where Z denotes a linear or branched hydrocarbon-based radical or a group corresponding to one of the following formulae:
—(CH2—CH2—O)x-CH2—CH2—
—[CH2—CH(CH3)-O]y-CH 2—CH(CH3)—
where x and y denote an integer from 1 to 4, representing a defined and unique degree of polymerization or any number from 1 to 4 representing an average degree of polymerization;
b) a bis-secondary diamine residue such as a piperazine derivative;
c) a bis-primary diamine residue of formula: —NH—Y—NH—, where Y denotes a linear or branched hydrocarbon-based radical, or alternatively the divalent radical
—CH2—CH2—S—S—CH2—CH2—;
d) a ureylene group of formula: —NH—CO—NH—;
Preferably, X⁻ is an anion such as chloride or bromide.
These polymers generally have a number-average molecular mass of between 1000 and 100 000.
Polymers of this type are described in particular in French patents 2 320 330, 2 270 846, 2 316 271, 2 336 434 and 2 413 907 and U.S. Pat. Nos. 2,273,780, 2,375,853, 2,388,614, 2,454,547, 3,206,462, 2,261,002, 2,271,378, 3,874,870, 4,001,432, 3,929,990, 3,966,904, 4,005,193, 4,025,617, 4,025,627, 4,025,653, 4,026,945 and 4,027,020.

Use may be made more particularly of polymers which are composed of repeating units conforming to the formula:

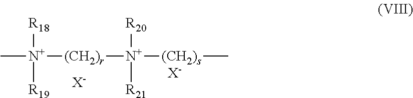

in which R18, R19, R20 and R21, which may be identical or different, denote an alkyl or hydroxyalkyl group having from about 1 to 4 carbon atoms, r and s are integers varying from 2 to 20 approximately, and X— is an anion derived from a mineral or organic acid.

One particularly preferred compound of formula (VIII) is that for which R18, R19, R20 and R21 represent a methyl radical and r=3, s=6 and X═Cl, which is called Hexadimethrine chloride according to the INCI (CTFA) nomenclature.

(8) Polyquaternary ammonium polymers composed of units of formula (IX):

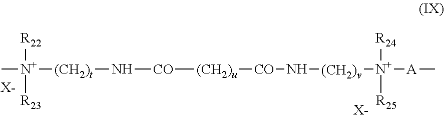

in which formula:
R22, R23, R24 and R25, which may be identical or different, represent a hydrogen atom or a methyl, ethyl, propyl, β-hydroxyethyl, β-hydroxypropyl or —CH2CH2(OCH2CH2)pOH radical,
in which p is equal to 0 or to an integer between 1 and 6, with the proviso that R22, R23, R24 and R25 do not simultaneously represent a hydrogen atom,
t and u, which may be identical or different, are integers between 1 and 6,
v is equal to 0 or to an integer between 1 and 34,
X— denotes an anion such as a halide,
A denotes a radical of a dihalide or represents preferably —CH2—CH2—O—CH2—CH2—.

Such compounds are especially described in patent application EP-A-122 324.

Among these products, examples that may be mentioned include Mirapol® A 15, Mirapol® AD1, Mirapol® AZ1 and Mirapol® 175 sold by the company Miranol.

(9) Quaternary polymers of vinylpyrrolidone and of vinylimidazole, for instance the products sold under the names Luviquat® FC 905, FC 550 and FC 370 by the company BASF.

(10) Cationic polysaccharides, especially cationic celluloses and galactomannan gums.

Among cationic polysaccharides, mention may be made more particularly of cellulose ether derivatives comprising quaternary ammonium groups, cationic cellulose copolymers or cellulose derivatives grafted with a water-soluble quaternary ammonium monomer and cationic galactomannan gums.

The cellulose ether derivatives comprising quaternary ammonium groups described in French patent 1 492 597. These polymers are also defined in the CTFA dictionary as quaternary ammoniums of hydroxyethylcellulose that have reacted with an epoxide substituted with a trimethylammonium group.

Cationic cellulose copolymers or cellulose derivatives grafted with a water-soluble monomer of quaternary ammonium, and described especially in U.S. Pat. No. 4,131,576, such as hydroxyalkylcelluloses, for instance hydroxymethyl-, hydroxyethyl- or hydroxypropylcelluloses grafted, in particular, with a methacryloylethyl-trimethylammonium, methacrylamidopropyltrimethylammonium or dimethyldiallylammonium salt.

The cationic galactomannan gums are described more particularly in U.S. Pat. Nos. 3,589,578 and 4,031,307, in particular guar gums containing cationic trialkylammonium groups. Guar gums modified with a salt (e.g. chloride) of 2,3-epoxypropyltrimethylammonium are used, for example.

Other cationic polymers that may be used in the context of the invention are cationic proteins or cationic protein hydrolysates, polyalkyleneimines, in particular polyethyleneimines, polymers containing vinylpyridine or vinylpyridinium units, condensates of polyamines and of epichlorohydrin, quaternary polyureylenes and chitin derivatives.

The cationic proteins or protein hydrolysates are, in particular, chemically modified polypeptides bearing quaternary ammonium groups at the end of the chain, or grafted thereon. Their molecular mass may vary, for example, from 1500 to 10 000 and in particular from 2000 to 5000 approximately. Among these compounds, mention may be made especially of:

Preferably, these polypeptides are of plant origin.

The following may especially be mentioned:
wheat, soybean or rice protein hydrolysates modified with cocodimonium hydroxypropyl groups,
wheat, soybean, jojoba, oat or rice protein hydrolysates modified with hydroxypropyl trimonium groups,
wheat, soybean or jojoba protein hydrolysates modified with lauridimonium hydroxypropyl groups,
jojoba, soybean or rice protein hydrolysates modified with steardimonium hydroxypropyl groups.

Among these products, an example that may be mentioned is the product sold by the company Cognis under the name Gluadin WQ or by the company Croda under the name Hydrotriticum WQ PE or Croquat Soya.

Among all the cationic polymers that may be used in the context of the present invention, it is preferred to use cationic cyclopolymers, in particular dimethyldiallylammonium chloride homopolymers or copolymers, sold under the names Merquat 100, Merquat 550 and Merquat S by the company Nalco, quaternary polymers of vinylpyrrolidone and of vinylimidazole, and cationic polysaccharides, and mixtures thereof.

The conditioning agent(s) that may be used according to the invention may be chosen from cationic surfactants.

The term “cationic surfactant” means a surfactant that is positively charged when it is contained in the composition according to the invention. This surfactant may bear one or more positive permanent charges or may contain one or more cationizable functions in the composition according to the invention.

The cationic surfactant(s) that may be used as conditioning agents according to the present invention are preferably chosen from primary, secondary or tertiary fatty amines, optionally polyoxyalkylenated, or salts thereof, and quaternary ammonium salts, and mixtures thereof.

The fatty amines generally comprise at least one C₈-C₃₀ hydrocarbon-based chain. Among the fatty amines that may be used according to the invention, examples that may be mentioned include stearylamidopropyldimethylamine and distearylamine.

Examples of quaternary ammonium salts that may especially be mentioned include:
those corresponding to the general formula (XII) below:

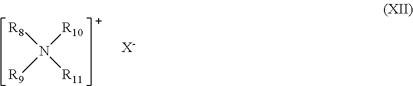

in which the radicals R₈ to R₁₁, which may be identical or different, represent a linear or branched aliphatic radical comprising from 1 to 30 carbon atoms or an aromatic radical such as aryl or alkylaryl, at least one of the radicals R₈ to R₁₁ comprising from 8 to 30 carbon atoms and preferably from 12 to 24 carbon atoms. The aliphatic radicals may comprise heteroatoms especially such as oxygen, nitrogen, sulfur and halogens. The aliphatic radicals are chosen, for example, from C₁₋₃₀ alkyl, C₁₋₃₀ alkoxy, polyoxyalkylene (C₂-C₈), C₁₋₃₀ alkylamide, (C₁₂-C₂₂)alkylamido(C₂-C₆)alkyl, (C₁₂-C₂₂)alkylacetate, and C₁₋₃₀ hydroxyalkyl; X⁻ is an anion chosen from the group of halides, phosphates, acetates, lactates, (C₁-C₄)alkyl sulfates and (C₁-C₄)alkyl- or (C₁-C₄)alkylaryl-sulfonates.

Among the quaternary ammonium salts of formula (XII), preference is given, on the one hand, to tetraalkylammonium chlorides, for instance or alkyltrimethylammonium chlorides in which the alkyl radical contains approximately 12 to 22 carbon atoms, in particular behenyltrimethylammonium chloride, distearyldimethylammonium chloride, cetyltrimethylammonium chloride and benzyldimethylstearylammonium chloride, or else, on the other hand, to palmitylamidopropyltrimethylammonium chloride or the stearamidopropyldimethyl(myristyl acetate)ammonium chloride sold under the name Ceraphyl® 70 by the company Van Dyk.
quaternary ammonium salts of imidazoline, for instance those of formula (XIII) below:

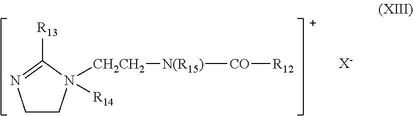

in which R₁₂ represents an alkyl or alkenyl radical comprising from 8 to 30 carbon atoms, for example fatty acid derivatives of tallow, R₁₃ represents a hydrogen atom, a C₁-C₄ alkyl radical or an alkyl or alkenyl radical comprising from 8 to 30 carbon atoms, R₁₄ represents a C₁-C₄ alkyl radical, R₁₅ represents a hydrogen atom or a C₁-C₄ alkyl radical, k is an anion chosen from the group of halides, phosphates, acetates, lactates, (C₁-C₄)alkyl sulfates and (C₁-C₄)alkyl- or (C₁-C₄)alkylaryl-sulfonates. Preferably, R₁₂ and R₁₃ denote a mixture of alkenyl or alkyl radicals comprising from 12 to 21 carbon atoms, for example fatty acid derivatives of tallow, R₁₄ denotes a methyl radical and R₁₅ denotes a hydrogen atom. Such a product is sold, for example, under the name Rewoquat®W 75 by the company Rewo;
quaternary di- or triammonium salts in particular of formula (XIV):

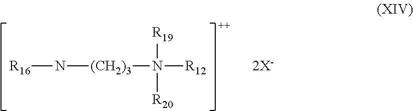

in which R16 denotes an alkyl radical comprising approximately 16 to 30 carbon atoms, optionally hydroxylated and/or interrupted with one or more oxygen atoms, R17 is chosen from hydrogen, an alkyl radical comprising from 1 to 4 carbon atoms and a group [(R16a)(R17a)(R18a)N⁺—(CH2)3-, Y], R16a, R17a, R18a, R18, R19, R20 and R21, which may be identical or different, are chosen from hydrogen and an alkyl radical comprising from 1 to 4 carbon atoms, and X⁻ and Y⁻, which may be identical or different, are halides, acetates, phosphates, nitrates, (C₁-C₄)alkyl sulfates, (C₁-C₄)alkyl-sulfonate or (C₁-C₄)alkylaryl-sulfonates, in particular methyl sulfate or ethyl sulfate. Such compounds are, for example, Finquat GT-P sold by the company Finetex (Quaternium-89) and Finquat CT sold by the company Finetex (Quaternium-75),
quaternary ammonium salts containing at least one ester function, such as those of formula (XV) below:

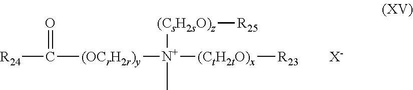

in which:

R₂₂ is chosen from C₁-C₆ alkyl and C₁-C₆ hydroxyalkyl or dihydroxyalkyl radicals;
R₂₃ is chosen from;
   the radical

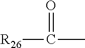

radicals R₂₇, which are linear or branched, saturated or unsaturated C₁-C₂₂ hydrocarbon-based radicals,
a hydrogen atom,
R₂₅ is chosen from:
   the radical

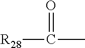

radicals R₂₉, which are linear or branched, saturated or unsaturated C₁-C₆ hydrocarbon-based radicals,
a hydrogen atom,

R₂₄, R₂₆ and R₂₈, which may be identical or different, are chosen from linear or branched, saturated or unsaturated C₇-C₂₁ hydrocarbon-based radicals;

r, s and t, which may be identical or different, are integers ranging from 2 to 6;

y is an integer ranging from 1 to 10;

x and z, which may be identical or different, are integers ranging from 0 to 10;

X⁻ is a simple or complex, organic or mineral anion;

with the proviso that the sum x+y+z is from 1 to 15, that when x is 0, then R₂₃ denotes R₂₇ and that when z is 0, then R₂₅ denotes R₂₉.

The alkyl radicals R₂₂ may be linear or branched, but more particularly linear.

R₂₂ preferably denotes a methyl, ethyl, hydroxyethyl or dihydroxypropyl radical, and more particularly a methyl or ethyl radical.

Advantageously, the sum x+y+z is from 1 to 10.

When R₂₃ is a hydrocarbon-based radical R₂₇, it may be long and may contain from 12 to 22 carbon atoms, or may be short and may contain from 1 to 3 carbon atoms.

When R₂₅ is a hydrocarbon-based radical R₂₉, it preferably contains 1 to 3 carbon atoms.

Advantageously, R₂₄, R₂₆ and R₂₈, which may be identical or different, are chosen from linear or branched, saturated or unsaturated C₁₁-C₂₁ hydrocarbon-based radicals, and more particularly from linear or branched, saturated or unsaturated C₁₁-C₂₁ alkyl and alkenyl radicals.

Preferably, x and z, which may be identical or different, are equal to 0 or 1.

Advantageously, y is equal to 1.

Preferably, r, s and t, which may be identical or different, are equal to 2 or 3, and even more particularly are equal to 2.

The anion X⁻ is preferably a halide (chloride, bromide or iodide), a (C₁-C₄)alkyl sulfate or a (C₁-C₄)alkyl- or (C₁-C₄)alkylaryl-sulfonate. However, methanesulfonate, phosphate, nitrate, tosylate, an anion derived from an organic acid, such as acetate or lactate, or any other anion that is compatible with the ammonium containing an ester function, may be used.

The anion X⁻ is even more particularly chloride, methyl sulfate or ethyl sulfate.

Use is made more particularly in the composition according to the invention of the ammonium salts of formula (XV) in which;
R₂₂ denotes a methyl or ethyl radical,
x and y are equal to 1;
z is equal to 0 or 1;
r, s and t are equal to 2;
R₂₃ is chosen from:
   the radical

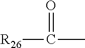

methyl, ethyl or C₁₄-C₂₂ hydrocarbon-based radicals,
a hydrogen atom;
R₂₅ is chosen from:
   the radical

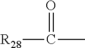

a hydrogen atom;
R₂₄, R₂₆ and R₂₈, which may be identical or different, are chosen from linear or branched, saturated or unsaturated C₁₃-C₁₇ hydrocarbon-based radicals and preferably from linear or branched, saturated or unsaturated C₁₃-C₁₇ alkyl and alkenyl radicals.

The hydrocarbon-based radicals are advantageously linear.

Examples that may be mentioned include the compounds of formula (XV) such as the diacyloxyethyldimethylammonium, diacyloxyethylhydroxyethylmethylammonium, monoacyloxyethyldihydroxyethylmethylammonium, triacyloxyethylmethylammonium and monoacyloxyethylhydroxyethyldimethylammonium salts (chloride or methyl sulfate in particular), and mixtures thereof. The acyl radicals preferably contain 14 to 18 carbon atoms and are obtained more particularly from a plant oil such as palm oil or sunflower oil. When the compound contains several acyl radicals, these radicals may be identical or different.

These products are obtained, for example, by direct esterification of triethanolamine, triisopropanolamine, an alkyldiethanolamine or an alkyldiisopropanolamine, which are optionally oxyalkylenated, with C10-C30 fatty acids or with mixtures of C10-C30 fatty acids of plant or animal origin, or by transesterification of the methyl esters thereof. This esterification is followed by a quaternization using an alkylating agent such as an alkyl halide (preferably a methyl or ethyl halide), a dialkyl sulfate (preferably dimethyl or diethyl sulfate), methyl methanesulfonate, methyl para-toluenesulfonate, glycol chlorohydrin or glycerol chlorohydrin.

Such compounds are sold, for example, under the names Dehyquart® by the company Henkel, Stepanquat® by the company Stepan, Noxamium® by the company CECA or Rewoquat® WE 18 by the company Rewo-Witco.

The composition according to the invention may contain, for example, a mixture of quaternary ammonium salts of mono-, di- and triesters with a weight majority of diester salts.

As mixture of ammonium salts, it is possible to use, for example, the mixture containing from 15% to 30% by weight of acyloxyethyldihydroxyethylmethylammonium methyl sulfate, from 45% to 60% of diacyloxyethylhydroxyethylmethylammonium methyl sulfate and from 15% to 30% of triacyloxyethylmethylammonium methyl sulfate, the acyl radicals having from 14 to 18 carbon atoms and originating from optionally partially hydrogenated palm oil.

It is also possible to use the ammonium salts containing at least one ester function that are described in U.S. Pat. Nos. US-A-4,874,554 and US-A-4,137,180.

The cationic surfactant(s) that may particularly preferably be used according to the invention are the compounds of formula (XII) or of formula (XV).

Among all the cationic surfactants that may be present in the composition according to the invention, the ones preferably chosen are cetyltrimethylammonium salts, behenyltrimethylammonium salts, dipalmitoylethylhydroxyethylmethylammonium salts, distearoylethylhydroxyethylmethylammonium salts, methyl(C₉-C₁₉)alkyl(C₁₀-C₂₀)alkylamidoethylimidazolium salts, the stearamidopropyldimethylamine salt and the stearamidopropyl dimethylammonium salt, and mixtures thereof.

Among the silicones that may be used as conditioning agents in accordance with the present invention, mention may be made, in a non-limiting manner, of:

I. Volatile Silicones:

These silicones have a boiling point of between 60° C. and 260° C. Among the silicones of this type that are mentioned are:

(i) cyclic silicones comprising from 3 to 7 and preferably 4 to 5 silicon atoms.

These are, for example, octamethylcyclotetrasiloxane sold under the name Volatile Silicone 7207® by the company Union Carbide or Silbione 70045 V2® by the company Rhône-Poulenc, decamethylcyclopentasiloxane sold under the name Volatile Silicone 7158® by the company Union Carbide, and Silbione 70045 V5® by the company Rhône-Poulenc, and mixtures thereof. Mention is also made of cyclopolymers of the dimethylsiloxane/methylalkylsiloxane type, such as Volatile Silicone FZ 3109® sold by the company Union Carbide, which is a dimethylsiloxane/methyloctylsiloxane cyclopolymer;

(ii) linear volatile silicones containing 2 to 9 silicon atoms and having a viscosity of less than or equal to 5×10⁻⁶ m²/s at 25° C.

Such a silicone is, for example, hexamethyldisiloxane sold under the name Silbione 70041 V0.65® by Rhône-Poulenc. This type of product is described in the article by Todd & Byers “Volatile silicone fluids for cosmetics”, Cosmetics and Toiletries, Vol. 91, January 76, pages 27 to 32.

II. Non-Volatile Silicones

These silicones are mainly constituted by polyalkylsiloxanes, polyarylsiloxanes, polyalkylarylsiloxanes and organomodified polysiloxanes, and mixtures thereof. They may be in the form of oils, gums and resins.

Among the polyalkylsiloxanes, mention may be made mainly of linear polydimethylsiloxanes with a viscosity of greater than 5×10⁻⁶ m²/s, and preferably less than 2.6 m²/s, i.e.:
containing trimethylsilyl end groups, for instance, in a non-limiting manner, the Silbione® oils of the 70047 series sold by the company Rhône-Poulenc, the oil Wacker Belsil DM 60000 from Wacker or certain Viscasil® products from the company General Electric,
containing trihydroxysilyl end groups of the 48 V® series from the company Rhône-Poulenc.

In this class of polyalkylsiloxanes, mention may also be made of the polyalkylsiloxanes sold by the company Goldschmidt, Abil Wax 9800® and Abil Wax 9801®, which are poly(C₁₋₂₀)alkylsiloxanes.

Among the polyalkylarylsiloxanes, mention may be made of linear and/or branched polydimethylphenylsiloxanes and polydimethyldiphenylsiloxanes, with a viscosity from 10⁻⁵ to 5×10⁻² m²/s, for instance:
the oil Rhodorsil® 763 from Rhône-Poulenc,
the Silbione® oils of the 70641 series from Rhône-Poulenc, such as the oils Silbione 70641 V30® and Silbione 70641 V200®,
the product DC 556® Cosmetic Grade Fluid from Dow Corning,
the silicones of the PK series from Bayer, such as PK20®,
the silicones of the PN and PH series from Bayer, such as the products PN 1000® and PH 1000®;
certain oils of the SF series from General Electric, such as SF 1250®, SF 1265®, SF 1154® and SF 1023®.

The silicone gums in accordance with the present invention are polydiorganosiloxanes with a high number-average molecular mass of between 200 000 and 1 000 000, used alone or as a mixture in a solvent chosen from volatile silicones, polydimethylsiloxane (PDMS) oils, polyphenylmethylsiloxane (PPMS) oils, isoparaffins, methylene chloride, pentane, dodecane, tridecane and tetradecane, or mixtures thereof.

Mention is made, for example, of compounds having the following structures:
poly[(dimethylsiloxane)/(methylvinylsiloxane)]gums,
poly[(dimethylsiloxane)/(diphenylsiloxane)]gums,
poly[(dihydrogenodimethylsiloxane)/(divinylsiloxane)]gums,
poly[(dimethylsiloxane)/(phenylmethylsiloxane)]gums,
poly[(dimethylsiloxane)/(diphenylsiloxane)/(methylvinylsiloxane)]gums.

Mention may be made of the gum Mirasil DM 300 000 from the company Rhodia.

Examples that may also be mentioned, in a non-limiting manner, include the following mixtures:

1) mixtures formed from a polydimethylsiloxane hydroxylated at the end of the chain (Dimethiconol according to the CTFA nomenclature) and of a polydimethylsiloxane (Cyclomethicone according to the CTFA nomenclature), such as the products Q2 1401® or Dow Corning 1501 Fluid sold by the company Dow Corning;

2) mixtures formed from a polydimethylsiloxane gum with a cyclic silicone, such as the product SF 1214 Silicone Fluid® from General Electric, which is a gum SE 30® of MW 500 000 (-Mₙ) dissolved in SF 1202 Silicone Fluid® (decamethylcyclopentasiloxane);

3) mixtures of two PDMSs of different viscosities, especially of a PDMS gum and a PDMS oil, such as the products SF 1236® and CF 1241® from the company General Electric. The product SF 1236® is a mixture of an SE 30® gum defined above, with a viscosity of 20 m²/s, and of an SF 96® oil with a viscosity of 5×10⁶ m²/s (15% SE 30® gum and 85% SF 96® oil).

The product CF 1241® is a mixture of an SE 30® gum (33%) and of a PDMS (67%), with a viscosity of 10⁻³ m²/s.

The organopolysiloxane resins that may be used in accordance with the invention are crosslinked siloxane systems containing the following units: R₂SiO_{2/2}, RSiO_{3/2} and SiO_{4/2} in which R represents a hydrocarbon group containing 1 to 6 carbon atoms or a phenyl group. Among these products, those particularly preferred are the ones in which R denotes a lower alkyl radical or a phenyl radical.

Among these resins, mention may be made of the product sold under the name Dow Corning 593® or those sold under the names Silicone Fluid SS 4230 and Silicone Fluid SS 4267 by the company General Electric, which are dimethyl/trimethylpolysiloxanes.

The organomodified silicones in accordance with the present invention are silicones as defined above, comprising in their general structure one or more organofunctional groups directly attached to the siloxane chain or attached via a hydrocarbon-based radical.

Mention is made, for example, of silicones comprising:

a) perfluoro groups such as trifluoroalkyls, for instance those sold by the company General Electric under the names FF.150 Fluorosilicone Fluid® or by the company Shin-Etsu under the names X-22-819®, X-22-820, X-22-821® and X-22-822®;

b) hydroxyacylamino groups, for instance those described in patent application EP 0 342 834 and in particular the silicone sold by the company Dow Corning under the name Q2-8413®;

c) thiol groups, as in the silicones X 2-8360® from the company Dow Corning or GP 72A® and GP 710 from Genesee;

d) non-quaternized amine groups, such as GP 4 Silicone Fluid® from Genesee, GP 7100® from Genesee, Q2 8220® from Dow Corning, AFL 40® from Union Carbide or the silicone known as Amodimethicone in the CTFA dictionary;

e) carboxylate groups, for instance the products described in patent EP 186 507 from Chisso Corporation;

f) hydroxylated groups, such as the polyorganosiloxanes containing a C2-C18 hydroxyalkyl function, described, for example, in patent application FR 85/16334.

Mention may be made most particularly of the product sold by Dow Corning under the name DC 190;

g) alkoxylated groups, for instance in the silicone copolymer F 755® from SWS Silicones and the products Abil Wax 2428®, Abil Wax 2434® and Abil Wax 2440® from the company Goldschmidt;

h) (C8-C22)acyloxy(C2-C18)alkyl groups, for instance the polyorganopolysiloxanes described in patent application FR 88/17433;

i) quaternary ammonium groups, as in the products X2 81 08® and X2 81 09® and the product Abil K 3270® from the company Goldschmidt;

j) amphoteric or betaine groups, as in the product sold by the company Goldschmidt under the name Abil B 9950®;

k) bisulfite groups, as in the products sold by the company Goldschmidt under the names Abil S 201® and Abil S 255®;

l) polyethyleneoxy and/or polypropyleneoxy groups optionally comprising C6-C24 alkyl groups, such as the products known as dimethicone copolyol sold by the company Dow Corning under the name DC 1248 or the oils Silwet L 722, L 7500, L 77 and L 711 by the company Union Carbide, and the (C12)alkylmethicone copolyol sold by the company Dow Corning under the name Q2 5200.

According to the invention, silicones comprising a polysiloxane portion and a portion formed from a non-silicone organic chain, one of the two portions forming the main chain of the polymer and the other being grafted onto the said main chain, may also be used. These polymers are described, for example, in patent applications EP-A-412 704, EP-A-412 707, EP-A-640 105 and WO 95/00578, EP-A-582 152 and WO 93/23009 and U.S. Pat. No. 4,693,935, U.S. Pat. No. 4,728,571 and U.S. Pat. No. 4,972,037. These polymers are preferably anionic or nonionic.

Such polymers are, for example, copolymers that may be obtained by radical polymerization from the monomer mixture formed from:

a) 50% to 90% by weight of tert-butyl acrylate;

b) 0% to 40% by weight of acrylic acid;

c) 5% to 40% by weight of a silicone macromer of formula:

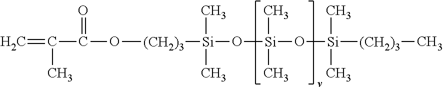

### (XVIII)

with v being a number ranging from 5 to 700; the weight percentages being calculated relative to the total weight of the monomers.

Other examples of grafted silicone polymers are especially polydimethylsiloxanes (PDMS) onto which are grafted, via a connecting unit of thiopropylene type, mixed polymer units of the poly(meth)acrylic acid type and of the polyalkyl(meth)acrylate type and polydimethylsiloxanes (PDMS) onto which are grafted, via a connecting unit of thiopropylene type, polymer units of the polyisobutyl(meth)acrylate type.

According to the invention, all the silicones may also be used in the form of emulsions, nanoemulsions or microemulsions.

The polyorganosiloxanes that are particularly preferred in accordance with the invention are:
non-volatile silicones chosen from the family of polydialkylsiloxanes containing trimethylsilyl end groups, such as oils with a viscosity of between 0.2 and 2.5 m²/s at 25° C., such as the oils of the series DC200 from Dow Corning, in particular the product with a viscosity of 60 000 cSt, of the series Silbione 70047, and more particularly the oil 70 047 V 500 000 sold by the company Rhodia Chimie, polydialkylsiloxanes containing dimethylsilanol end groups such as dimethiconols, or polyalkylarylsiloxanes such as the oil Silbione 70641 V 200 sold by the company Rhodia Chimie;
polysiloxanes containing amine groups such as amodimethicones or trimethylsilyl amodimethicones.

The viscosities of the silicones may especially be determined by the standard ASTM D445-97 (viscometry).

When the conditioning agent of the composition according to the invention is a hydrocarbon, it is a linear or branched C₈-C₃₀₀ hydrocarbon.

Among the hydrocarbons that are liquid at room temperature corresponding to this definition, mention may be made especially of isododecane, isohexadecane and isomers thereof (such as 2,2,4,4,6,6-heptamethylnonane), isoeicosane, isotetracosane, isomers of the said compounds, n-nonadecane, n-dodecane, n-undecane, n-tridecane and n-pentadecane, and mixtures of these hydrocarbons. Use is preferably made according to the invention of isododecane or an isomer thereof.

When the conditioning agent is a fatty alcohol, it is of the linear or branched, saturated or unsaturated C8-C30 type. Among the latter agents, examples that may be mentioned include 2-butyloctanol lauryl alcohol, oleyl alcohol, 2-octyldodecanol, isocetyl alcohol, isostearyl alcohol and behenyl alcohol, and mixtures thereof.

When the conditioning agent is a fatty ester, it may be either an ester of a C₈-C₃₀ fatty acid and of a C₁-C₃₀ alcohol including esters of a C₈-C₃₀ fatty acid and of C₈-C₃₀ fatty alcohols, or an ester of a C₁-C₇ acid or diacid and of a C₈-C₃₀ fatty alcohol.

Among these esters, examples that may be mentioned include ethyl, isopropyl, 2-ethylhexyl and 2-octyldecyl palmitate, isopropyl, butyl, cetyl and 2-octyldecyl myristate, butyl and hexyl stearate, hexyl and 2-hexyldecyl laurate, isononyl isononanoate, dioctyl malate, dioctyl, myristyl myristate, stearyl myristate, cetyl palmitate, myristyl stearate, stearyl stearate and cetyl stearate, and mixtures thereof.

The ceramides or ceramide analogues, such as glycoceramides, which may be used as conditioning agents in the compositions according to the invention are known per se and are natural or synthetic molecules that may correspond to the general formula (XIX) below:

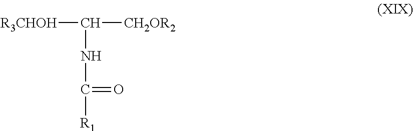

in which:
R₁ denotes a linear or branched, saturated or unsaturated alkyl radical, derived from C₁₄-C₃₀ fatty acids, this radical possibly being substituted with a hydroxyl group in the alpha position or a hydroxyl group in the omega position esterified with a saturated or unsaturated C₁₆-C₃₀ fatty acid;
R₂ denotes a hydrogen atom or a radical (glycosyl)ₙ, (galactosyl)ₘ or sulfogalactosyl, in which n is an integer ranging from 1 to 4 and m is an integer ranging from 1 to 8;
R₃ denotes a saturated or unsaturated C₁₅-C₂₆ hydrocarbon-based radical in the alpha position, this radical possibly being substituted with one or more C₁-C₁₄ alkyl radicals;
it being understood that, in the case of these natural ceramides or glycoceramides, R₃ may also denote a C₁₅-C₂₆ alpha-hydroxyalkyl radical, the hydroxyl group being optionally esterified with a C₁₆-C₃₀ alpha-hydroxyacid.

The ceramides that are preferred in the context of the present invention are those described by Downing in Arch. Dermatol., Vol. 123, 1381-1384, 1987, or those described in French patent FR 2 673 179.

The ceramide(s) that are more particularly preferred according to the invention are the compounds for which R₁ denotes a saturated or unsaturated alkyl derived from C₁₆-C₂₂ fatty acids; R₂ denotes a hydrogen atom; and R₃ denotes a saturated linear C₁₅ radical.

Such compounds are, for example:
N-linoleoyldihydrosphingosine,
N-oleoyldihydrosphingosine,
N-palmitoyldihydrosphingosine,
N-stearoyldihydrosphingosine,
N-behenoyldihydrosphingosine,

or mixtures of these compounds.

Even more preferentially, use is made of ceramides for which R₁ denotes a saturated or unsaturated alkyl radical derived from fatty acids; R₂ denotes a galactosyl or sulfogalactosyl radical; and R₃ denotes a group —CH═CH—(CH₂)₁₂—CH₃.

By way of example, mention may be made of the product formed from a mixture of these compounds, sold under the trade name Glycocer by the company Waitaki International Biosciences.

Among all these conditioning agents, used is preferably made of one or more conditioning agents chosen from silicones and cationic polymers.

The cosmetic composition according to the invention preferably comprises at least 0.01% by weight and more preferentially from 0.05% to 10% by weight of conditioning agent(s), relative to the total weight of the composition.

The cosmetic composition according to the invention may have a pH of between 3 and 10 and preferentially between 5 and 7. This pH may be adjusted by means of acidifying and basifying agents conventionally used in cosmetics.

The cosmetic composition used according to the invention may also contain one or more adjuvants that are common in cosmetics, such as hair-loss counteractants, oxidizing agents, vitamins and provitamins including panthenol, plant, animal, mineral or synthetic oils, waxes, sunscreens, mineral or organic, coloured or uncoloured pigments, dyes, nacreous agents and opacifiers, sequestrants, plasticizers, solubilizers, antioxidants, hydroxy acids, fragrances, antidandruff agents other than the compounds (i) and (ii) as defined previously, and preserving agents. The adjuvants(s) that may be used in the cosmetic composition of the invention are different from the compounds defined previously.

The amounts of these various adjuvants are those conventionally used in the fields under consideration.

Needless to say, a person skilled in the art will take care to select the optional compound(s) to be added to the composition according to the invention, such that the advantageous properties intrinsically associated with the composition in accordance with the invention are not, or are not substantially, adversely affected by the envisaged addition.

Another subject of the invention consists of a cosmetic antidandruff treatment process, for improving and/or reducing dandruff, and especially that caused by yeasts of the genus Malassezia, characterized in that it comprises the application of a cosmetically effective amount of a composition according to the invention to the hair and the scalp. The composition may then be optionally rinsed out with water.

A subject of the invention is also the use of the combination of at least one compound (i) as defined above and of at least one compound (ii) as defined previously, preferably in a weight ratio of the amount of compound(s) (i) to the amount of compound(s) (ii) of greater than or equal to 0.5, preferably greater than or equal to 0.7 and better still greater than or equal to 1, as an antidandruff agent. The use of the said combination makes it possible to eliminate and/or reduce dandruff on the hair and the scalp while limiting the irritation and itching of the scalp.

Finally, a subject of the invention is the use of the combination of at least one compound (i) as defined above and of at least one compound (ii) as defined previously, preferably in a weight ratio of the amount of compound(s) (i) to the amount of compound(s) (ii) of greater than or equal to 0.5, preferably greater than or equal to 0.7 and better still greater than or equal to 1, as an anti-canities agent.

The examples that follow are intended to illustrate the invention, without, however, having any limiting nature.

Example of Preparation of the Bacterial Extract:

The process for preparing a biomass containing the bacterial strain Vitreoscilla filiformis comprises the culturing of the bacteria in an oxygenated sterile medium, in the presence of mineral salts and sugars; the harvesting; and then centrifugation, of the culture medium comprising the bacteria, to obtain a biomass, which is placed in bottles and then sterilized. Rupture of the cells resulting from the sterilization causes decantation of the biomass as a sludge essentially containing cell membranes and coagulated proteins. A homogeneous biomass is reconstituted by agitation before use.

The following compositions were prepared according to the following tables. The amounts are indicated as weight percentages of active material (AM) relative to the total weight of each composition.

### EXAMPLE 1

### Leave-On Aqueous-Alcoholic Gel

| Composition 1 | weight % of AM |
|---|---|
| Crosslinked carboxyvinyl | 0.3 |
| homopolymer (Carbopol Ultrez 10 | |
| Polymer from the company Lubrizol) | |
| 96° ethyl alcohol | 17.4 |
| Ellagic acid | 0.5 |
| Extract of Vitreoscilla filiformis as an | 0.25 |
| aqueous dispersion containing 5% | |
| solids, prepared according to the | |
| method indicated above (1) | |
| 2-amino-2-methyl-1-propanol | qs pH 7.0 |
| Water | qs 100 |

The gel obtained is stable over time and has satisfactory rheology.

When applied to the scalp and the hair, this gel shows good cosmetic performance qualities, in particular a good feel and a good antidandruff effect associated with a notable reduction of irritation and itching.

### EXAMPLE 2

### Antidandruff Shampoo

| Composition 2 | weight % of AM |
|---|---|
| Cocamidopropylbetaine as an aqueous solution | 2.4 |
| containing 38% by weight of active material | |
| (Tegobetaine F50 from Evonik Goldschmidt) | |
| Sodium benzoate | 0.5 |
| Sodium lauryl ether sulfate containing 2.2 mol of | 15.54 |
| ethylene oxide, at 26% by weight in aqueous | |
| solution (Texapon AOS 225 UP sold by the | |
| company Cognis) | |
| Extract of Vitreoscilla filiformis as an aqueous | 0.5 |
| dispersion containing 5% active material, prepared | |
| according to the method indicated above (1) | |
| Ellagic acid | 1 |
| 1-(Hexadecyloxy)-2-octadecanol/cetyl alcohol | 2.5 |
| mixture (47/53 by weight) | |
| Salicylic acid | 0.2 |
| Coconut monoisopropanolamide (Comperlan 100 | 0.25 |
| sold by the company Cognis) | |
| Polydimethylsiloxane (Mirasil DM 500 000 sold by | 2 |
| the company Rhodia) | |
| Propylene glycol | 0.1 |
| Salicylic acid | 0.2 |
| Glycerol | 2 |
| Fragrance | 0.5 |
| Carboxyvinyl polymer (Carbopol 980 Polymer sold | 0.3 |
| by the company Lubrizol) | |
| Sodium chloride | 1.1 |
| pH agent qs | pH 5.3 |
| Water | qs 100 |

The shampoo obtained is stable over time and has satisfactory rheology.

When applied to the hair and the scalp and rinsed out, this shampoo has good working qualities (ease of application, soft, pleasant lather, ease of removal), good cosmetic performance qualities (softness, smoothness) and a satisfactory antidandruff effect, in particular on repeated application, associated with a notable reduction of irritation and itching.

1-16. (canceled)

17. A cosmetic composition comprising:
(i) from about 0.01% to about 10% by weight, relative to the total weight of the composition, at least one compound chosen from ellagic acid, ethers of ellagic acid, ellagic acid salts, and ethers of ellagic acid salts;
(ii) at least one extract of non-photosynthetic, non-fruiting filamentous bacteria; and
(iii) at least one surfactant chosen from anionic, amphoteric, and zwitterionic surfactants.

18. The composition according to claim 17, wherein the at least one compound (i) is chosen from alkali metal or alkaline-earth metal salts of ellagic acid and ethers thereof.

19. The composition according to claim 17, wherein the at least one compound (i) is chosen from sodium, potassium, calcium, and magnesium salts of ellagic acid and ethers thereof.

20. The composition according to claim 17, wherein the at least one compound (i) is an ellagic acid ether or ellagic acid salt ether chosen from mono-, di-, tri- or polyethers obtained by etherification of one or more hydroxyl groups of ellagic acid with one or more groups chosen from C₂-C₂₀ alkyl groups, polyoxyalkylene groups and groups derived from one or more mono- or polysaccharides.

21. The composition according to claim 17, wherein the at least one compound (i) is ellagic acid or a salt thereof.

22. The composition according to claim 17, wherein the at least one compound (i) is present in a an amount ranging from about 0.1% to about 5% by weight, relative to the total weight of the composition.

23. The composition according to claim 17, wherein the at least one compound (i) is present in a an amount ranging from about 0.2% to about 2% by weight, relative to the total weight of the composition.

24. The composition according to claim 17, wherein the at least one non-photosynthetic, non-fruiting filamentous bacterial extract is an extract of Vitreoscilla filiformis.

25. The composition according to claim 17, wherein the at least one non-photosynthetic, non-fruiting filamentous bacteria extract is present in an amount ranging from about 0.001% to about 10% by weight, expressed as solids relative to the total weight of the composition.

26. The composition according to claim 17, wherein the at least one non-photosynthetic, non-fruiting filamentous bacteria extract is present in an amount ranging from about 0.01% to about 5% by weight, expressed as solids relative to the total weight of the composition.

27. The composition according to claim 17, wherein the weight ratio of the at least one compound (i)/the at least one extract (ii) is greater than or equal to 0.5.

28. The composition according to claim 17, wherein the weight ratio of the at least one compound (i)/the at least one extract (ii) is greater than or equal to 0.7.

29. The composition according to claim 17, wherein the weight ratio of the at least one compound (i)/the at least one extract (ii) is greater than or equal to 1.

30. The composition according to claim 17, further comprising at least one thickener.

31. The composition according to claim 17, further comprising at least one conditioning agent.

32. The composition according to claim 17, further comprising at least one conditioning agent chosen from silicones and cationic polymers.

33. The composition according to claim 17, further comprising at least one additional component chosen from hair-loss counteractants, oxidizing agents, vitamins and provitamins including panthenol, plant, animal, mineral or synthetic oils, waxes, sunscreens, mineral or organic, coloured or uncoloured pigments, dyes, nacreous agents and opacifiers, sequestrants, plasticizers, solubilizers, acidifying agents, basifying agents, antioxidants, hydroxy acids, fragrances, antidandruff agents, and preserving agents.

34. A method of eliminating and/or reducing dandruff, said method comprising applying, to the head and scalp, an effective amount of a composition comprising, in a cosmetically acceptable medium:
(i) from about 0.01% to about 10% by weight, relative to the total weight of the composition, at least one compound chosen from ellagic acid, ethers of ellagic acid, ellagic acid salts, and ethers of ellagic acid salts;
(ii) at least one extract of non-photosynthetic, non-fruiting filamentous bacteria; and
(iii) at least one surfactant chosen from anionic, amphoteric, and zwitterionic surfactants.

35. A method of improving and/or imparting anti-dandruff and/or anti-canities properties in a cosmetic composition, said method comprising adding an effective amount of the following components to said cosmetic composition:
(i) at least one compound chosen from ellagic acid, ethers of ellagic acid, ellagic acid salts, and ethers of ellagic acid salts; and
(ii) at least one extract of non-photosynthetic, non-fruiting filamentous bacteria.
